(19) United States
(12) Patent Application Publication
(10) Pub. No.: US 2015/0275252 A1
(43) Pub. Date: Oct. 1, 2015

(54) PRODUCTION OF FERMENTABLE BIOMASS SUGARS USING HIGH-SOLIDS ENZYMATIC HYDROLYSIS

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US); Ryan ZEBROSKI, Fayetteville, GA (US)

(21) Appl. No.: 14/670,457

(22) Filed: Mar. 27, 2015

Related U.S. Application Data
(63) Continuation-in-part of application No. 14/583,572, filed on Dec. 26, 2014.
(60) Provisional application No. 61/971,138, filed on Mar. 27, 2014.

(51) Int. Cl. C12P 19/04; C12P 7/56; C12P 7/04; C12P 7/54; C12P 19/02; C12P 7/10

(52) U.S. Cl. CPC C12P 19/04; C12P 19/02; C12P 7/10; C12P 7/04; C12P 7/54; C12P 7/56

(57) ABSTRACT

In some variations, this invention provides a process for producing fermentable sugars from cellulosic biomass, comprising: extracting biomass with steam or hot water to produce an extract liquor containing hemicellulose oligomers, dissolved lignin, and cellulose-rich solids; separating and washing the cellulose-rich solids; removing a portion of glucan contained in the washed cellulose-rich solids as glucose oligomers using a liquefaction-focused blend of enzymes; co-hydrolyzing glucose oligomers and hemicellulose oligomers, with enzymes or an acid catalyst, to produce glucose and hemicellulose monomers; and recovering the glucose and hemicellulose monomers as fermentable sugars. The liquefaction-focused blend of enzymes contains endoglucanases and exoglucanases. A rotating unit for high-solids enzymatic hydrolysis may be employed, with continuous liquid removal. Optionally, the glucose and the hemicellulose monomers may be recovered as separate streams. The residual cellulose (not hydrolyzed) as well as the lignin may be recovered and combusted, or utilized for other purposes.

### PRIORITY DATA

This patent application is a non-provisional application claiming priority to U.S. Provisional Patent App. No. 61/971,138, filed Mar. 27, 2014, and further is a continuation-in-part application of U.S. patent application Ser. No. 14/583,572, filed Dec. 26, 2014, each of which is hereby incorporated by reference herein.

### FIELD OF THE INVENTION

The present invention generally relates to processes for preparing fermentable sugars from lignocellulosic biomass.

### BACKGROUND OF THE INVENTION

The Green Power+® technology has been developed by American Process, Inc. This technology extracts hemicelluloses from a biomass feedstock supply and converts only those hemicelluloses into sugars which are then fermented, such as to cellulosic ethanol. Green Power+ technology is a two-step process to produce sugars from hemicelluloses. An initial steam or hot-water extraction pulls out hemicelluloses, and the remainder of the biomass (cellulose/lignin) is not exposed to any acidic treatment. The remaining solids remain suitable for combustion in a boiler or for pelletization, or other uses. The extracted solution is then hydrolyzed with a mild acid or enzyme treatment to hydrolyze oligomers into fermentable monomers.

The biomass that has been extracted of hemicelluloses is suitable for a variety of downstream applications, including combustion in biomass boilers, combined heat and power, torrefaction, pelleting, pulping, or production of specialty products (e.g. panels). Co-location with a biomass power plant leads to synergies and cost advantages.

What are desired are variations of the Green Power+ technology that target or allow high yields of fermentable sugars, including from some of the cellulose portion of the starting biomass feedstock.

### SUMMARY OF THE INVENTION

The present invention addresses the aforementioned needs in the art.

In some variations, the invention provides a process for producing fermentable sugars from cellulosic biomass, the process comprising:

(a) providing a feedstock comprising cellulosic biomass;

(b) extracting the feedstock with an extraction solution including steam and/or hot water under effective extraction conditions to produce an extract liquor containing hemicellulose oligomers, dissolved lignin, and cellulose-rich solids;

(c) separating at least a portion of the cellulose-rich solids from the extract liquor, to produce washed cellulose-rich solids;

(d) removing a portion of glucan contained in the washed cellulose-rich solids by contacting the washed cellulose-rich solids with a liquefaction-focused blend of enzymes, to release glucose oligomers;

(e) co-hydrolyzing the glucose oligomers and the hemicellulose oligomers, with enzymes or an acid catalyst, to produce glucose and hemicellulose monomers; and

(f) recovering the glucose and hemicellulose monomers as fermentable sugars.

In some embodiments, the extraction solution comprises steam in saturated, superheated, or supersaturated form. In some embodiments, the extraction solution comprises hot water.

In some embodiments, step (c) includes washing the cellulose-rich solids using an aqueous wash solution, to produce a wash filtrate; and optionally combining at least some of the wash filtrate with the extract liquor. In some of these embodiments, step (c) further includes pressing the cellulose-rich solids to produce the washed cellulose-rich solids and a press filtrate; and optionally combining at least some of the press filtrate with the extract liquor. Step (c) may include countercurrent washing, such as in two or more washing stages.

In some embodiments, step (d) (application of enzymes) is conducted between a first and second washing stage. In some embodiments, step (d) is conducted following a second washing stage. Step (d) may be integrated with step (c), and in certain embodiments, step (c) and step (d) are conducted in a single unit. The process may further comprise refining or milling the washed cellulose-rich solids prior to or during step (d).

In some embodiments, the liquefaction-focused blend of enzymes in step (d) includes endoglucanases and exoglucanases. In some embodiments, the enzymes in step (e) include cellulases and hemicellulases.

The process further comprises a step of fermenting the fermentable sugars to a fermentation product, in some embodiments.

Other variations provide a process for producing fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulose oligomers, dissolved lignin, and cellulose-rich solids;

(c) separating at least a portion of the cellulose-rich solids from the extract liquor, to produce washed cellulose-rich solids;

(d) removing a portion of glucan contained in the washed cellulose-rich solids by contacting the washed cellulose-rich solids with a liquefaction-focused blend of enzymes, to release glucose oligomers;

(e) hydrolyzing the glucose oligomers with a first hydrolysis catalyst, to produce glucose;

(f) hydrolyzing the hemicellulose oligomers with a second hydrolysis catalyst, to produce hemicellulose monomers; and

(g) recovering the glucose and hemicellulose monomers, individually or in combination, as fermentable sugars.

In some embodiments, the first hydrolysis catalyst includes cellulases. In some embodiments, the second hydrolysis catalyst includes hemicellulases. In other embodiments, the first hydrolysis catalyst and the second hydrolysis catalyst are acid catalysts. The first hydrolysis catalyst may be the same as, or different than, the second hydrolysis catalyst.

In some embodiments, the glucose is recovered in a separate stream from the hemicellulose monomers. In other embodiments, the glucose and the hemicellulose monomers are recovered in the same stream. The process may include fermentation of the glucose and/or the fermentable hemicellulose sugars to a fermentation product.

In any of these processes, the feedstock may include sucrose. When the starting biomass material contains sucrose, it may be present in a concentration of (for example) from about 0.5 wt % to about 10 wt % sucrose, or from about 1 wt % to about 5 wt % sucrose. In some embodiments with sucrose present in the feedstock, a majority of the sucrose is recovered as part of the fermentable sugars.

In some embodiments, a rotating unit for high-solids enzymatic hydrolysis is employed. The rotating unit may contain internal means, such as metal balls or other objects, for pressing solids. In some embodiments, the rotating unit contains a screen or other means for continuous liquid removal. In certain embodiments, a rotary kiln is retrofitted for high-solids enzymatic hydrolysis.

### BRIEF DESCRIPTION OF THE FIGURES

FIG. 1 is a simplified block-flow diagram depicting the process of some embodiments of the present invention, producing a combined fermentable sugars stream.

FIG. 2 is a simplified block-flow diagram depicting the process of certain embodiments of the present invention, producing a fermentable hemicellulose sugars stream and a glucose stream.

### DETAILED DESCRIPTION OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

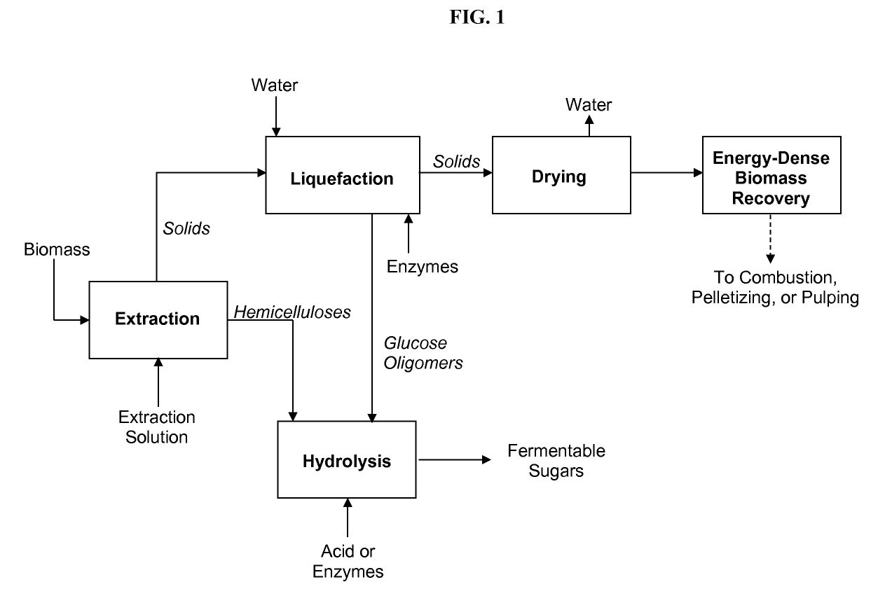

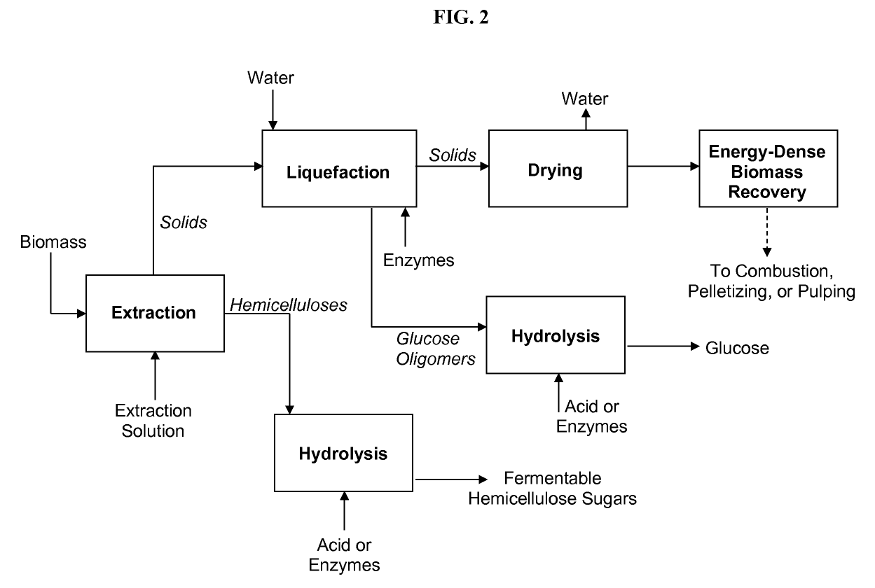

Certain exemplary embodiments of the invention will now be described, including by reference to exemplary FIG. 1 and FIG. 2. These embodiments are not intended to limit the scope of the invention as claimed. The order of steps may be varied, some steps may be omitted, and/or other steps may be added. Reference herein to first step, second step, etc. is for illustration purposes only.

In some variations (including FIG. 1), the invention provides a process for producing fermentable sugars from cellulosic biomass, the process comprising:

(a) providing a feedstock comprising cellulosic biomass;

(b) extracting the feedstock with an extraction solution including steam and/or hot water under effective extraction conditions to produce an extract liquor containing hemicellulose oligomers, dissolved lignin, and cellulose-rich solids;

(c) separating at least a portion of the cellulose-rich solids from the extract liquor, to produce washed cellulose-rich solids;

(d) removing a portion of glucan contained in the washed cellulose-rich solids by contacting the washed cellulose-rich solids with a liquefaction-focused blend of enzymes, to release glucose oligomers;

(e) co-hydrolyzing the glucose oligomers and the hemicellulose oligomers, with enzymes or an acid catalyst, to produce glucose and hemicellulose monomers; and

(f) recovering the glucose and hemicellulose monomers as fermentable sugars.

In some embodiments, the extraction solution comprises steam in saturated, superheated, or supersaturated form. In some embodiments, the extraction solution comprises hot water. Additives may be present, such as acid or base catalysts, or other compounds present in recycled streams.

In some embodiments, step (c) includes washing the cellulose-rich solids using an aqueous wash solution, to produce a wash filtrate; and optionally combining at least some of the wash filtrate with the extract liquor. In some of these embodiments, step (c) further includes pressing the cellulose-rich solids to produce the washed cellulose-rich solids and a press filtrate; and optionally combining at least some of the press filtrate with the extract liquor.

Step (c) may include countercurrent washing, such as in two, three, four, or more washing stages. Step (d) may be integrated with step (c), and in certain embodiments, step (c) and step (d) are conducted in a single unit. That is, the separation/washing in step (c) may be combined with the application of the liquefaction-focused blend of enzymes in step (d), in various ways.

The application of the liquefaction-focused blend of enzymes may be conducted prior to a first washing stage, during (integrated with) a first washing stage, between a first and second washing stage, during (integrated with) a second washing stage, after a second washing stage, or during (integrated with) or after a later washing stage.

In some embodiments, the liquefaction-focused blend of enzymes in step (d) includes both endoglucanases and exoglucanases. Endoglucanases are cellulases that attack low-crystallinity regions in the cellulose fibers by endoaction, creating free chain-ends; exoglucanases or cellobiohydrolases are cellulases that hydrolyze the 1,4-glycocidyl linkages to form cellobiose.

Other cellulase enzymes may be utilized as recited in Verardi et al., “Hydrolysis of Lignocellulosic Biomass: Current Status of Processes and Technologies and Future Perspectives,” Bioethanol, Prof Marco Aurelio Pinheiro Lima (Ed.), ISBN: 978-953-51-0008-9, InTech (2012), which is hereby incorporated by reference.

Thermotolerant enzymes may be employed, in various embodiments of the invention. For example, in some embodiments, a suitable enzyme is utilized at high temperature for only hydrolysis and little or no saccharification (i.e., no production of glucose monomer) and then the temperature is decreased after which the enzyme may also accomplish saccharification.

The process may further comprise refining or milling the washed cellulose-rich solids prior to or during step (d).

When step (e) employs enzymes, these enzymes will typically contain cellulases and hemicellulases. The cellulases here may include β-glucosidases that convert cellooligosaccharides and disaccharide cellobiose into glucose. There are a number of enzymes that can attack hemicelluloses, such as glucoronide, acetylesterase, xylanase, β-xylosidase, galactomannase and glucomannase. Exemplary acid catalysts for step (e) include sulfuric acid, sulfur dioxide, hydrochloric acid, phosphoric acid, and nitric acid.

The process further comprises a step of fermenting the fermentable sugars to a fermentation product, in some embodiments.

Other variations (such as FIG. 2) provide a process for producing fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulose oligomers, dissolved lignin, and cellulose-rich solids;

(c) separating at least a portion of the cellulose-rich solids from the extract liquor, to produce washed cellulose-rich solids;

(d) removing a portion of glucan contained in the washed cellulose-rich solids by contacting the washed cellulose-rich solids with a liquefaction-focused blend of enzymes, to release glucose oligomers;

(e) hydrolyzing the glucose oligomers with a first hydrolysis catalyst, to produce glucose;

(f) hydrolyzing the hemicellulose oligomers with a second hydrolysis catalyst, to produce hemicellulose monomers; and

(g) recovering the glucose and hemicellulose monomers, individually or in combination, as fermentable sugars.

In some embodiments, the first hydrolysis catalyst includes cellulases. In some embodiments, the second hydrolysis catalyst includes hemicellulases. In other embodiments, the first hydrolysis catalyst and the second hydrolysis catalyst are acid catalysts. The first hydrolysis catalyst may be the same as, or different than, the second hydrolysis catalyst.

In some embodiments, the glucose is recovered in a separate stream from the hemicellulose monomers. In other embodiments, the glucose and the hemicellulose monomers are recovered in the same stream. The process may include fermentation of the glucose and/or the fermentable hemicellulose sugars to a fermentation product.

The biomass feedstock may be selected from hardwoods, softwoods, forest residues, agricultural residues (such as sugarcane bagasse), industrial wastes, consumer wastes, or combinations thereof. In any of these processes, the feedstock may include sucrose. When the starting biomass material contains sucrose, it may be present in a concentration of (for example) from about 0.5 wt % to about 10 wt % sucrose, or from about 1 wt % to about 5 wt % sucrose. In some embodiments with sucrose present in the feedstock, a majority of the sucrose is recovered as part of the fermentable sugars. In order to preserve sucrose, it is preferred to utilize enzymes rather than acid catalysts for cellulose hydrolysis.

In some embodiments, such as the process depicted in FIG. 1, the process starts as biomass is received or reduced to approximately ¼″ thickness. In a first step of the process, the biomass chips are fed to a pressurized extraction vessel operating continuously or in batch mode. The chips may be steamed or water-washed to remove dirt and entrained air. The chips are immersed with aqueous liquor or saturated vapor and heated to a temperature between about 100° C. to about 250° C., for example 150° C., 160° C., 170° C., 180° C., 190° C., 200° C., or 210° C. Preferably, the chips are heated to about 180° C. to 210° C. The pressure in the pressurized vessel may be adjusted to maintain the aqueous liquor as a liquid, a vapor, or a combination thereof. Exemplary pressures are about 1 atm to about 30 atm, such as about 3 atm, 5 atm, 10 atm, or 15 atm.

The aqueous liquor may contain acidifying compounds, such as (but not limited to) sulfuric acid, sulfurous acid, sulfur dioxide, acetic acid, formic acid, or oxalic acid, or combinations thereof. The dilute acid concentration can range from 0.01% to 10% as necessary to improve solubility of particular minerals, such as potassium, sodium, or silica. Preferably, the acid concentration is selected from about 0.01% to 4%, such as 0.1%, 0.5%, 1%, 1.5%, 2%, 2.5%, 3%, or 3.5%.

A second step may include depressurization of the extracted chips. The vapor can be used for heating the incoming woodchips or cooking liquor, directly or indirectly. The volatilized organic acids (e.g., acetic acid), which are generated or included in the cooking step, may be recycled back to the cooking.

A third step may include washing the extracted chips. The washing may be accomplished with water, recycled condensates, recycled permeate, or combination thereof. A liquid biomass extract is produced. A countercurrent configuration may be used to maximize the biomass extract concentration. Washing typically removes most of the dissolved material, including hemicelluloses and minerals. The final consistency of the dewatered cellulose-rich solids may be increased to 30% or more, preferably to 50% or more, using a mechanical pressing device.

The third step, or an additional step prior to drying, may include further hydrolyzing the extracted chips with a liquefaction-focused blend of enzymes to convert some of the cellulose to glucose oligomers. In some preferred embodiments, the additional hydrolysis is mild hydrolysis that leaves a substantial portion of cellulose in the extracted solids. The mild hydrolysis can take advantage of the initial extraction (first step) of most or all of the hemicellulosic material, leaving a somewhat hollow structure. The hollow structure can increase the effectiveness of cellulose hydrolysis, such as by reducing mass-transfer limitations of enzymes or acids in solution.

When enzymes are employed for the cellulose hydrolysis, the enzymes are preferably cellulase enzymes. Enzymes may be introduced to the extracted chips along with the wash solution, e.g. water, recycled condensates, recycled permeate, or combinations thereof. Alternatively, or additionally, enzymatic hydrolysis may be carried out following washing and removal of hemicelluloses, minerals, and other soluble material.

Enzymes may be added to the extracted chips before or after mechanical pressing. That is, enzymatic hydrolysis may be carried out and then the solids pressed to final consistency; or, the solids may be pressed to high consistency (e.g., 30% or more) and then enzymes introduced to carry out cellulose hydrolysis. It may be beneficial to conduct refining or milling of the dewatered cellulose-rich solids prior to the enzymatic hydrolysis.

The enzymatic hydrolysis may be achieved in a separate unit, such as between washing and drying, or as an integrated part of washing. In some embodiments, at least a portion of enzymes are recycled in a batch or continuous process.

Some embodiments of the invention are premised on the use of rotating apparatus so that cellulose-rich solids and enzymes may be combined and mixed at high solids consistency. For example, a unit similar to a cement mixer may be utilized. Optionally, metal balls or another means of mechanical pressing may be included in the rotating unit. Also, the unit may be configured with a screen for continuous liquid removal (the liquid containing hydrolyzed sugars in monomer or oligomer form). The solids consistency may be about 10 wt %, 15 wt %, 20 wt %, 25 wt %, 30 wt %, 35 wt %, 40 wt %, 45 wt %, 50 wt % or higher, for example. The rotation may be performed continuously or periodically.

In certain embodiments, a rotary kiln (such as a lime kiln) is retrofitted to be used for enzymatic hydrolysis.

When an acid is employed for the cellulose hydrolysis, the acid may be selected from sulfuric acid, sulfurous acid, sulfur dioxide, formic acid, acetic acid, oxalic acid, or combinations thereof. Dilute-acid hydrolysis is preferred, to avoid sugar degradation. Acids may be introduced to the extracted chips along with the wash solution, e.g. water, recycled condensates, recycled permeate, or combinations thereof. Alternatively, or additionally, acid hydrolysis may be carried out following washing and removal of hemicelluloses, minerals, and other soluble material.

Acids may be added to the extracted chips before or after mechanical pressing. That is, acid hydrolysis may be carried out and then the solids pressed to final consistency; or, the solids may be pressed to high consistency (e.g., 30% or more) and then acids introduced to carry out cellulose hydrolysis. It may be beneficial to conduct refining or milling of the dewatered cellulose-rich solids prior to the acid hydrolysis.

The acid hydrolysis may be achieved in a separate unit, such as between washing and drying, or as an integrated part of washing. In some embodiments, at least a portion of the acid is recycled in a batch or continuous process.

A fourth step may include drying of the extracted material to a desired final moisture. The heat necessary for drying may be derived from combusting part of the starting biomass. Alternatively, or additionally, the heat for drying may be provided by other means, such as a natural gas boiler or other auxiliary fossil fuel, or from a waste heat source.

A fifth step may include preparing the biomass for combustion. This step may include refining, milling, fluidizing, compacting, and/or pelletizing the dried, extracted biomass. The biomass may be fed to a boiler in the form of fine powder, loose fiber, pellets, briquettes, extrudates, or any other suitable form. Using known equipment, biomass may be extruded through a pressurized chamber to form uniformly sized pellets or briquettes.

A sixth step may include treatment of the biomass extract to form a hydrolysate comprising fermentable hemicellulose sugars. In some embodiments, the biomass extract is hydrolyzed using dilute acidic conditions at temperatures between about 100° C. and 190° C., for example about 120° C., 130° C., 140° C., 150° C., 160° C., or 170° C., and preferably from 120° C. to 150° C.

The acid may be selected from sulfuric acid, sulfurous acid, or sulfur dioxide. Alternatively, or additionally, the acid may include formic acid, acetic acid, or oxalic acid from the cooking liquor or recycled from previous hydrolysis. Alternatively, hemicellulase enzymes may used instead of acid hydrolysis. The lignin from this step may be separated and recovered, or recycled to increase the heating value of the pellets, or sent directly to the boiler.

A seventh step may include evaporation of hydrolysate to remove some or most of the volatile acids. The evaporation may include flashing or stripping to remove sulfur dioxide, if present, prior to removal of volatile acids. The evaporation step is preferably performed below the acetic acid dissociation pH of 4.8, and most preferably a pH selected from about 1 to about 2.5. The dissolved solids are concentrated, such as to about 10% to about 40% to optimize fermentable hemicellulose sugar concentration to a particular microorganism. Saccharomyces Cerevisiae fermentation can withstand dissolved solids concentrations of 30-50%, while Clostridia Acetobutylicum fermentation is viable at 10-20% concentrations only, for example.

In some embodiments, additional evaporation steps may be employed. These additional evaporation steps may be conducted at different conditions (e.g., temperature, pressure, and pH) relative to the first evaporation step.

In some embodiments, some or all of the organic acids evaporated may be recycled, as vapor or condensate, to the first step (cooking step) and/or third step (washing step) to remove assist in the removal of minerals from the biomass. This recycle of organic acids, such as acetic acid, may be optimized along with process conditions that may vary depending on the amount recycled, to improve the cooking and/or washing effectiveness.

Some embodiments of the invention enable processing of “agricultural residues,” which for present purposes is meant to include lignocellulosic biomass associated with food crops, annual grasses, energy crops, or other annually renewable feedstocks. Exemplary agricultural residues include, but are not limited to, corn stover, corn fiber, wheat straw, sugarcane bagasse, rice straw, oat straw, barley straw, miscanthus, energy cane, or combinations thereof. In certain embodiments, the agricultural residue is sugarcane bagasse.

In some embodiments, the fermentable hemicellulose sugars are recovered from solution, in purified form. In some embodiments, the fermentable hemicellulose sugars are fermented to produce of biochemicals or biofuels such as (but by no means limited to) ethanol, 1-butanol, isobutanol, acetic acid, lactic acid, or any other fermentation products. A purified fermentation product may be produced by distilling the fermentation product, which will also generate a distillation bottoms stream containing residual solids. A bottoms evaporation stage may be used, to produce residual solids.

Following fermentation, residual solids (such as distillation bottoms) may be recovered, or burned in solid or slurry form, or recycled to be combined into the biomass pellets. Use of the fermentation residual solids may require further removal of minerals. Generally, any leftover solids may be used for burning as additional liquefied biomass, after concentration of the distillation bottoms.

Part or all of the residual solids may be co-combusted with the energy-dense biomass, if desired. Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

Optionally, the process may include co-combusting the recovered lignin with the energy-dense biomass, to produce power. The recovered lignin may be combined with the energy-dense biomass prior to combustion, or they may be co-fired as separate streams. When recovered lignin is combined with the energy-dense biomass for making pellets, the lignin can act as a pellet binder.

Part or all of the residual solids may be co-combusted with the energy-dense biomass, if desired. Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

In certain embodiments, the process further comprises combining, at a pH of about 4.8 to 10 or higher, a portion of the vaporized acetic acid with an alkali oxide, alkali hydroxide, alkali carbonate, and/or alkali bicarbonate, wherein the alkali is selected from the group consisting of potassium, sodium, magnesium, calcium, and combinations thereof, to convert the portion of the vaporized acetic acid to an alkaline acetate. The alkaline acetate may be recovered. If desired, purified acetic acid may be generated from the alkaline acetate.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing fermentable sugars from cellulosic biomass, said process comprising:
(a) providing a feedstock comprising cellulosic biomass;
(b) extracting said feedstock with an extraction solution including steam and/or hot water under effective extraction conditions to produce an extract liquor containing hemicellulose oligomers, dissolved lignin, and cellulose-rich solids;
(c) separating at least a portion of said cellulose-rich solids from said extract liquor, to produce washed cellulose-rich solids;
(d) removing a portion of glucan contained in said washed cellulose-rich solids by contacting said washed cellulose-rich solids with a liquefaction-focused blend of enzymes, to release glucose oligomers;
(e) co-hydrolyzing said glucose oligomers and said hemicellulose oligomers, with enzymes or an acid catalyst, to produce glucose and hemicellulose monomers; and
(f) recovering said glucose and hemicellulose monomers as fermentable sugars.

2. The process of claim 1, wherein said extraction solution comprises steam in saturated, superheated, or supersaturated form.

3. The process of claim 1, wherein said extraction solution comprises hot water.

4. The process of claim 1, wherein step (c) includes washing said cellulose-rich solids using an aqueous wash solution, to produce a wash filtrate; and optionally combining at least some of said wash filtrate with said extract liquor.

5. The process of claim 4, wherein step (c) further includes pressing said cellulose-rich solids to produce said washed cellulose-rich solids and a press filtrate; and optionally combining at least some of said press filtrate with said extract liquor.

6. The process of claim 1, wherein step (c) comprises countercurrent washing.

7. The process of claim 6, wherein said countercurrent washing is conducted in two or more washing stages.

8. The process of claim 7, wherein step (d) is conducted between a first and second washing stage.

9. The process of claim 7, wherein step (d) is conducted following a second washing stage.

10. The process of claim 1, wherein step (d) is integrated with step (c).

11. The process of claim 10, wherein step (c) and step (d) are conducted in a single unit.

12. The process of claim 1, wherein said liquefaction-focused blend of enzymes includes endoglucanases and exoglucanases.

13. The process of claim 1, said process further comprising refining or milling said washed cellulose-rich solids prior to or during step (d).

14. The process of claim 1, wherein said enzymes in step (e) include cellulases and hemicellulases.

15. The process of claim 1, said process further comprising a step of fermenting said fermentable sugars to a fermentation product.

16. The process of claim 1, wherein a rotating unit for high-solids enzymatic hydrolysis is employed.

17. The process of claim 16, wherein said rotating unit contains internal means for pressing solids.

18. The process of claim 16, wherein said rotating unit contains a screen or other means for continuous liquid removal.

19. The process of claim 1, wherein a rotary kiln is employed for high-solids enzymatic hydrolysis.

20. A process for producing fermentable sugars from cellulosic biomass, said process comprising:
(a) providing a feedstock comprising cellulosic biomass;
(b) extracting said feedstock with an extraction solution including steam and/or hot water under effective extraction conditions to produce an extract liquor containing hemicellulose oligomers, dissolved lignin, and cellulose-rich solids;
(c) separating at least a portion of said cellulose-rich solids from said extract liquor, to produce washed cellulose-rich solids;
(d) removing a portion of glucan contained in said washed cellulose-rich solids by contacting said washed cellulose-rich solids with a liquefaction-focused blend of enzymes, to release glucose oligomers;
(e) hydrolyzing said glucose oligomers with a first hydrolysis catalyst, to produce glucose;
(f) hydrolyzing said hemicellulose oligomers with a second hydrolysis catalyst, to produce hemicellulose monomers; and
(g) recovering said glucose and hemicellulose monomers, individually or in combination, as fermentable sugars,
wherein a rotating unit for high-solids enzymatic hydrolysis is employed.
